(19) United States
(12) Patent Application Publication
(10) Pub. No.: US 2021/0398638 A1
(43) Pub. Date: Dec. 23, 2021

(54) DOSING CALCULATOR FOR SPASTICITY

(71) Applicant: IPSEN BIOPHARM LIMITED, Wrexham (GB)

(72) Inventor: Leonor VIGNOL, Wrexham (GB)

(21) Appl. No.: 17/309,249

(22) PCT Filed: Nov. 25, 2019
(86) PCT No.: PCT/GB2019/053321

§ 371 Date: May 12, 2021

Related U.S. Application Data
(60) Provisional application No. 62/917,220, filed on Nov. 27, 2018.

(51) Int. Cl. G16H 20/17; G16H 40/67

(52) U.S. Cl. CPC G16H 20/17; G16H 40/67

(57) ABSTRACT

Mobile computing application for use in the determination of a recommended dose of a medicinal. Computer-implemented method for determining a dose for the administration of a medicinal to a subject wherein information is input by a user and the computer determines the total close for administration. Method for treating a disorder in a subject in need of such treatment, the method comprising using a computer-implemented method for determining a dose of the medicinal to be administered to the subject and administering such a dose to the subject.

### FIELD OF THE INVENTION

The present invention relates to a mobile computing application (“app”) for use in the determination of a dose of a medicinal for use in treating a disorder in a subject. The invention also relates to a computer-implemented method for determining such a dose. The invention further relates to a method for treating a disorder in a subject in need of such treatment, the method comprising using a computer-implemented method for determining a dose of the medicinal to be administered to the subject and administering such a dose to the subject.

### BACKGROUND OF THE INVENTION

It is well understood in the relevant art that particular disorders in a subject may be treated using a variety of medicinal substances (hereinafter “medicinals”). Such medicinals include pharmaceutical agents, dietary supplements, home remedies, herbal remedies, and the like.

Such medicinals may be administered by any of a variety of known means, for example, by ingestion, topical application, inhalation, injection, and transdermal administration.

In each such case, it is typical that a particular dose and/or dosing regimen of the medicinal is needed to treat the subject. Even for the same medicinal, however, the recommended dose may vary based on variables such as the particulars of the subject, the particulars of the disorder, the location for the desired administration, and the like.

Accordingly, a need exists for a dosing calculator app and a computer-implemented method that allow for the determination of a recommended dose and/or dosing regimen for a medicinal. A need also exists for a method of treatment wherein a dose determined using such an app and/or such a method is administered to a subject in need of such treatment.

### BRIEF DESCRIPTION OF THE DRAWINGS

FIGS. 1-16 illustrate an embodiment of the present invention related to a dosing calculator app for use in determining the recommended dose of DYSPORT® to administer to a subject.

### DETAILED DESCRIPTION OF THE DISCLOSURE

The figures and descriptions provided herein may have been simplified to illustrate aspects that are relevant for a clear understanding of the present invention, while eliminating, for the purpose of clarity, other aspects that may be found in typical similar devices, systems, and methods. Those of ordinary skill may thus recognize that other elements and/or operations may be desirable and/or necessary to implement the invention. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Embodiments are provided throughout so that this disclosure is sufficiently thorough and fully conveys the scope of the disclosed embodiments to those skilled in the art. Numerous specific details are set forth, such as examples of specific components, devices, and methods, to provide a thorough understanding of embodiments of the present disclosure. Nevertheless, it will be apparent to those skilled in the art that certain specific disclosed details need not be employed, and that embodiments may be embodied in different forms. As such, the embodiments should not be construed to limit the scope of the disclosure. As referenced above, in some embodiments, well-known processes, well-known device structures, and well-known technologies may not be described in detail.

The terminology used herein is for the purpose of describing particular embodiments only and is not intended to be limiting. For example, as used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The steps, processes, and operations described herein are not to be construed as necessarily requiring their respective performance in the particular order discussed or illustrated, unless specifically identified as a preferred or required order of performance. It is also to be understood that additional or alternative steps may be employed, in place of or in conjunction with the disclosed aspects.

Further, although the terms first, second, third, etc. may be used herein to describe various elements, components, regions, layers and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the embodiments.

The present invention relates to a mobile computing application (“app”) for use in the determination of a dose and/or dosing regimen of a medicinal for use in treating a disorder in a subject. The app may be used on a mobile device.

As used herein, a mobile device provides processing capabilities suitable to execute non-transitory computing code in performance of the one or more functions described herein. A mobile device may operate over one or more networks, such as including, but not limited to, cellular networks, wi-fi networks, bluetooth, mesh networks, infrared, and the like.

Such networking capability of the mobile device providing the app disclosed herein may communicatively connect the mobile device that includes the app to one or more backend processor systems. These one or more backend processors may, by way of non-limiting example, comprise networked servers in communication remotely with the one or more apps on the mobile device, wherein the backend servers may share processing duties with the mobile device. For example, the backend servers may allow for the mobile device to perform as a “thin client”, wherein the mobile device relies appreciably on the remote network server for processing capabilities, or as a “thick client”, wherein the mobile device provides the bulk of the processing capabilities and performance of the functions described herein.

The application may provide an interface whereby the user is enabled to input information regarding the medicinal to be administered.

An interface may also be provided whereby the user is enabled to input the disorder to be treated.

An interface may further be provided whereby the user is enabled to input information regarding the subject's characteristics, for example the subject's age, gender, weight, disorder, and the like.

An interface wherein the user is enabled to input the type of administration that will be performed, for example parenteral, transdermal, oral, and the like, may also be provided. In cases wherein the administration may occur at different dosing frequencies and/or with different dosing regimens, the user may be enabled to select a dosing frequency and/or dosing regimen. In cases wherein the administration may optionally be at more than one site, the user may also be enabled to input how many sites the administration will be at (e.g. unilateral or bilateral injection).

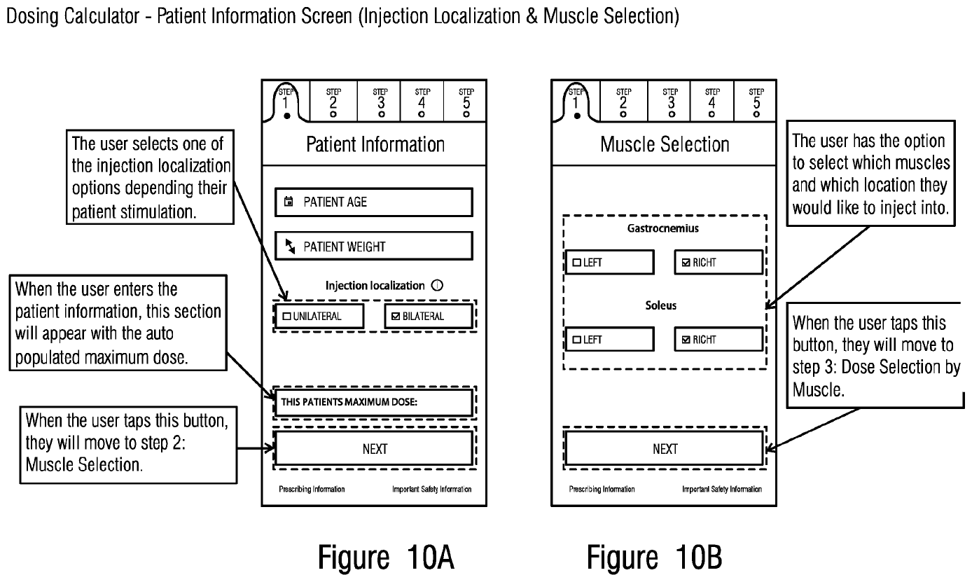

In instances wherein the administration may be at particular sites, the user may be enabled to input the site or sites at which the medicinal is to be administered (see e.g., FIG. 10B).

Based on the user's input respecting the medicinal, subject's characteristics, the type of administration, and/or location(s) of administration, a dose or dosing regimen is determined. Such a determination may be based on the recommended doses or dosing regimens known in the art for the selected medicinal given the subject's characteristics, the type of administration, and or location(s) of administration. The determination may be accomplished by means, for example algorithms, known in the art. The determined dose(s) or dosing regimen may then be displayed on a screen.

In certain instances wherein administration may be at two or more sites and a total recommended dose is provided, the user may be enabled to input a desired dose or dosing regimen for one or more of the sites.

In a particular such embodiment, based on the desired dose or dosing regimen for one or more of the sites, the parameters within which the desired dose or dosing regimen for the remaining site(s) may be limited. For example, if a total dose for a bilateral administration must be below a certain amount and a particular dose amount for a first site has been chosen, the dose amount for the second site may be limited so that the total of the two doses falls within the range for the total dose amount. In such an embodiment, the user may, for example, be precluded from selecting a dose amount for the second site that would cause the total dose amount to exceed its recommended range. For example, a screen may display scales which the user may adjust (e.g., by sliding) to indicate the desired dose at a location. The user's input on a first scale may limit the parameters within which the second scale may be adjusted.

In the alternative or in addition, a warning may appear on the screen advising the user that a selected dose amount is below or exceeds the recommended range or would cause the total dose amount to exceed such a range.

In another particular embodiment, based on the desired dose or dosing regimen for one or more of the sites, a recommended dose or dosing regimen for the remaining site(s) may be determined. In an example wherein the screen displays scales which the user may adjust to indicate the desired dose at a location, the user's input(s) on one or more such scales may cause the remaining scale(s) to automatically adjust to indicate a desired dose at a second location.

It is also contemplated that the parameters of a dose amount may be pre-set by an overseeing physician or body or the like.

An interface whereby the user is enabled to input the type of container, for example the size of a vial, from which the medicinal is to be supplied may also be provided. Based on the total dose for the subject, the number of containers for use in administering the medicinal to the subject is determined. For example, if the total dose for the subject is 250 units and the user selects a 100 unit vial as the preferred container, it will be determined that three 100 unit vials should be used.

The app may additionally provide an interface whereby the user is enabled to input a preferred concentration or dilution for the medicinal. Based on the user's input, the total volume for injection may be determined.

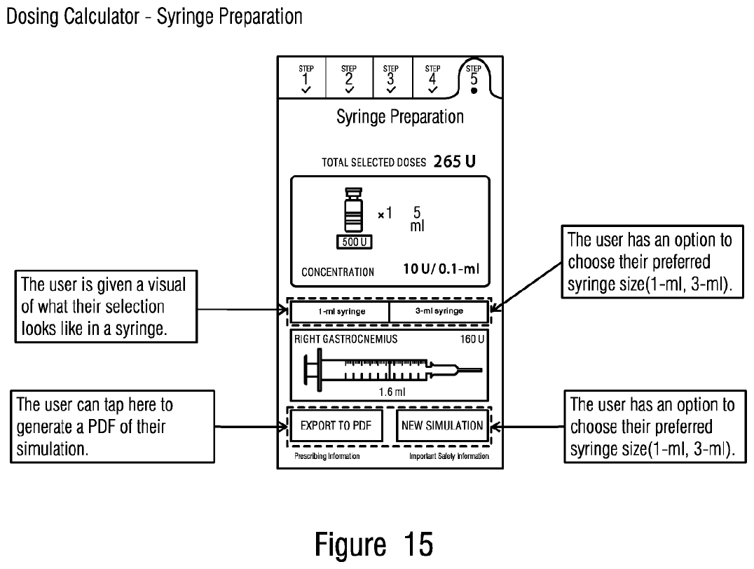

An interface may be provided whereby the user is enabled to input the type of administration device, for example a syringe, to be used. Based on the input, the app may display how the medicinal will appear in the syringe (see e.g. FIG. 15).

The user may be enabled to receive all or some of the information displayed by the app in a document. The document may, for example, be a PDF or the like. The document may also be printed or forwarded, for example in an e-mail.

The present invention contemplates that all or some of the above interfaces and displays may appear on the same screen, that each may appear on different screens, or may appear in a pop-up display on a screen.

In an embodiment wherein the interfaces and/or displays appear on two or more screens, the user may navigate between the screens by means known in the art, for example by swiping in a particular direction or by tapping a button. In the alternative, the app may automatically advance from a first screen to another once the requisite input for the interface(s) on a first screen has been completed.

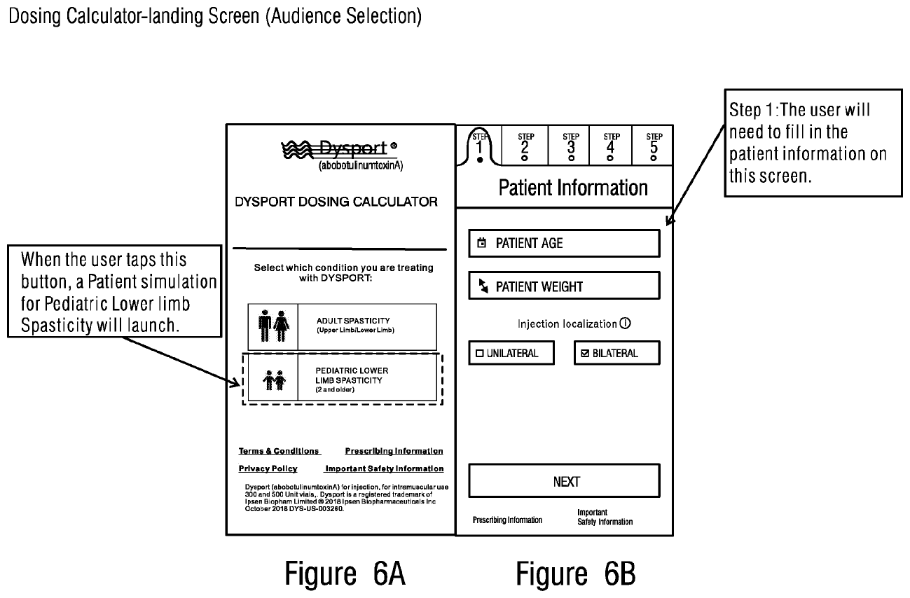

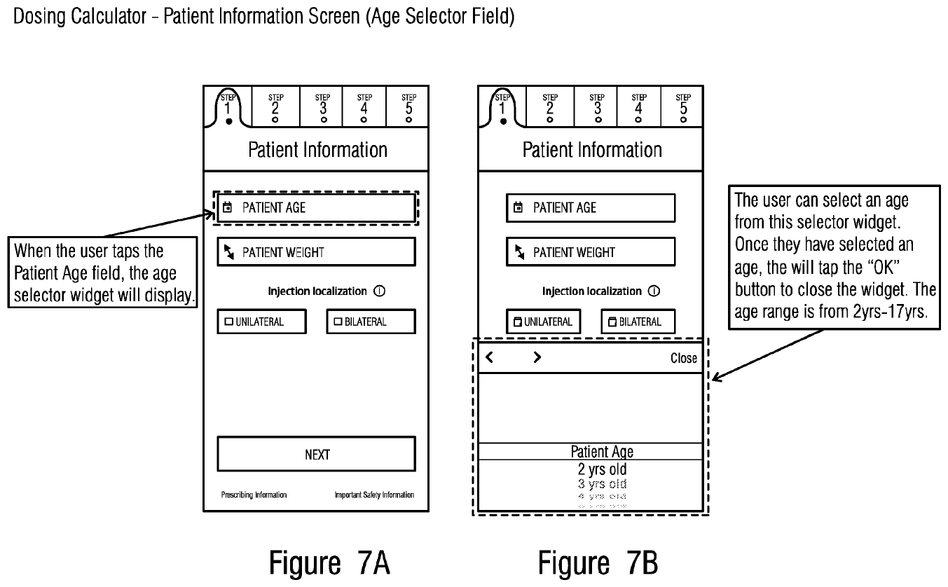

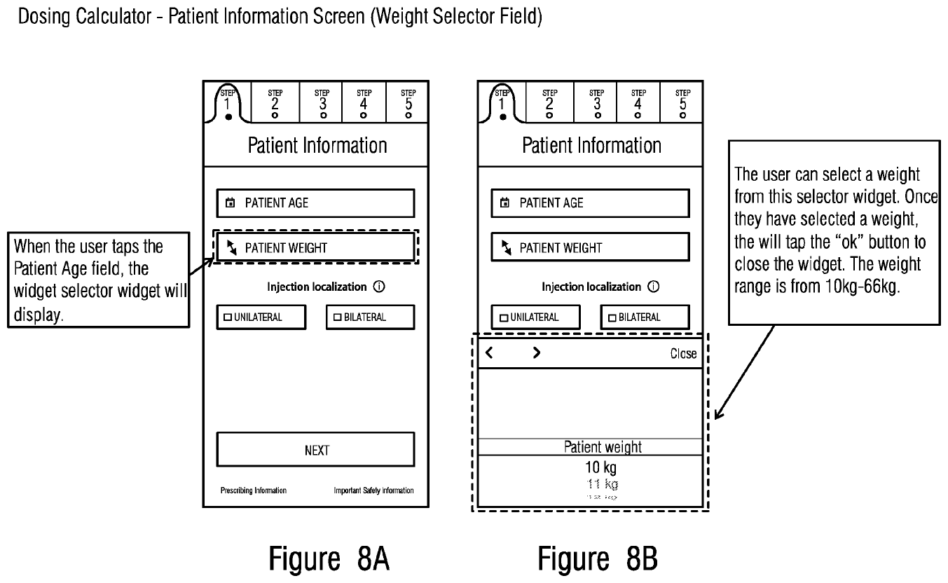

The input of information by the user may be conducted by any means known in the art. For example, the user may enter such information by direct input (e.g. typing the requisite input), tapping a button illustrated on the screen (see e.g. FIG. 6A), selection using a selection widget (see e.g. FIGS. 7B and 8B), or tapping on a check-box (see eg. FIGS. 10A-B).

In certain embodiments, what may be entered may be limited, based on the information previously provided, for example, the medicinal to be used, the patient's age, the patient's weight, etc. For example, if the subject is a pediatric patient, only ages below 18 may be entered.

In certain embodiments, such a limitation may also serve as an indication of what entries are proper. For example, a selector widget for a pediatric patient may include only selectable weights from 10 kg to 66 kg. Thereby, the app may be indicating that a child heavier than 66 kg should be qualified as an adult patient for purposes of dose calculation.

The app may also display information, for example a warning, dosing information, legal information, terms and conditions, privacy policies, and the like. In certain embodiments, the user may be required to acknowledge that he/she has read the information and/or accepted the terms displayed. Such acknowledgement may be made by the various means of input discussed previously.

The user may be enabled to open or close a display and/or zoom in or out of a display. Such may be accomplished by means known in the art.

The invention contemplates that the app allows for aspects of typical app functionality. For example, the app may display a link which, when tapped, advances the user to a webpage or to a display. In addition, the ability to return to a previous screen; save, download, print, or engage in other functionality; and the capability to tap, pinch, or otherwise access zooming in and zooming out features, may be provided.

Of note with regard to the disclosed embodiments, use of the app as a thick or thin client, such as is described above, may allow for access by a user of the app to a vast wealth of anonymized dosing and use information. As such, intercommunication regarding dosing, which may greatly improve dosing calculation, may be provided by the embodiments.

The subject may be anyone who is to be treated for a disorder by the administration of the medicinal. For example, the subject may be a patient suffering from the disorder.

The user may be any individual, including a medical professional such as the subject's doctor, or the subject him/herself. The user may also be an individual that is neither a medical professional nor a subject to be treated but rather an individual that wishes to determine a recommended dose for a medicinal for research purposes or for simple curiosity.

The app may be intended for use only by certain users, such as health care professionals only, and/or users in a certain country. Accordingly, a warning may accompany the downloading of the app and/or the downloading and/or use of the app may require entry of credentials.

Of course, it will be appreciated that certain aspects of the app may also be made available to the subject. Thus, the invention also contemplates a “patient-facing” version of the disclosed app that allows the subject to access those aspects.

In an embodiment, the determined dose(s) and dosing regimen(s) may be accessed by a third party, for example a health care professional.

The present invention also relates to a computer-implemented method for determining a dose of a medicinal for use in treating a disorder in a subject. The method comprises a step or steps wherein information relating to the subject, the medicinal to be administered, and/or the location(s) of such administration is input. Based on such information, a total dose amount is determined.

In an embodiment, the administration may be at more than one location. In such an embodiment, the dose amount at each location may be determined. In the alternative, the method may further contain a step wherein the dose to be administered at each location is selected. The parameters of what may be selected may be limited based on the information previously provided.

The method may also further contain steps wherein the type of container (e.g., vial) and/or the dilution for the medicinal is/are selected. Based on such information, the number of containers for use in each administration and the total volume of the medicinal formulation for administration may be determined.

Additionally, the method may contain steps wherein the method of administration and/or the administration device (e.g. syringe, transdermal patch, etc.) is/are selected. Based on such information, an illustration may be generated displaying the format of the administration, for example a syringe filled with the requisite amount of the formulation.

The present invention further relates to a method for treating a disorder in a subject in need of such treatment, the method comprising using a computer-implemented method, such as that described previously, for determining a dose of the medicinal to be administered to the subject and administering such a dose to the subject.

In embodiments of this treatment method, the computer-implemented method for determining a dose of the medicinal may be performed by a mobile computing application, such as that described previously.

The computer-implemented method and/or the mobile computing application performing such a method may be used by a first party for the purpose of determining a dose of the medicament for administration. Administration may then be performed by either that first party or a second party. Administration may also be performed by the subject him/herself.

The disorder to be treated in the embodiments of the present invention may be any disorder known to be treatable or ameliorated using a medicinal. For example, the disorder may be a movement disorder such as spasticity. In particular embodiments, the disorder is upper and/or lower limb spasticity in an adult or lower limb spasticity in a pediatric subject.

The medicinal for which a dose or dosing regimen is to be determined and/or which is to be administered to the subject may be any medicinal known to treat or ameliorate a disorder in a subject. For example, the medicinal may be a pharmaceutical agent, a dietary supplement, a home remedy, or a herbal remedy. In certain embodiments, the medicinal is a neurotoxin, such as a botulinum neurotoxin. In a particular embodiment, the medicinal is botulinum neurotoxin A, such as DYSPORT®.

The administration of the medicinal may be by any means known to administer a medicinal to a subject. For example, administration may be oral, topical, by inhalation, parenteral, buccal, transdermal, intramuscular, subcutaneous, intravenous, intradermal, etc.

In a particular embodiment of the present invention, the medicinal is botulinum neurotoxin, for example, botulinum toxin A (BoNT/A). BoNT/A is available from Ipsen (DYSPORT®, Ipsen Limited, Slough, UK). The disorders to be treated include adult upper and/or lower limb spasticity and pediatric lower limb spasticity.

In an embodiment of the mobile computing app for using in determining doses of DYSPORT®) for administration to a patient suffering from spasticity, the information to be input by the user includes the subject's age and weight, whether administration is to be unilateral or bilateral, and which muscle(s) is/are to be administered to. In this embodiment, the app may determine a total dose amount and allow the user to select the amounts for administration to each muscle. The app may also allow the user to select the vial to be used for the administration of DYSPORT® and the dilution to be used. Based on such information, the app will determine the total volume for administration. The app may also allow the user to choose the syringe used for such administration.

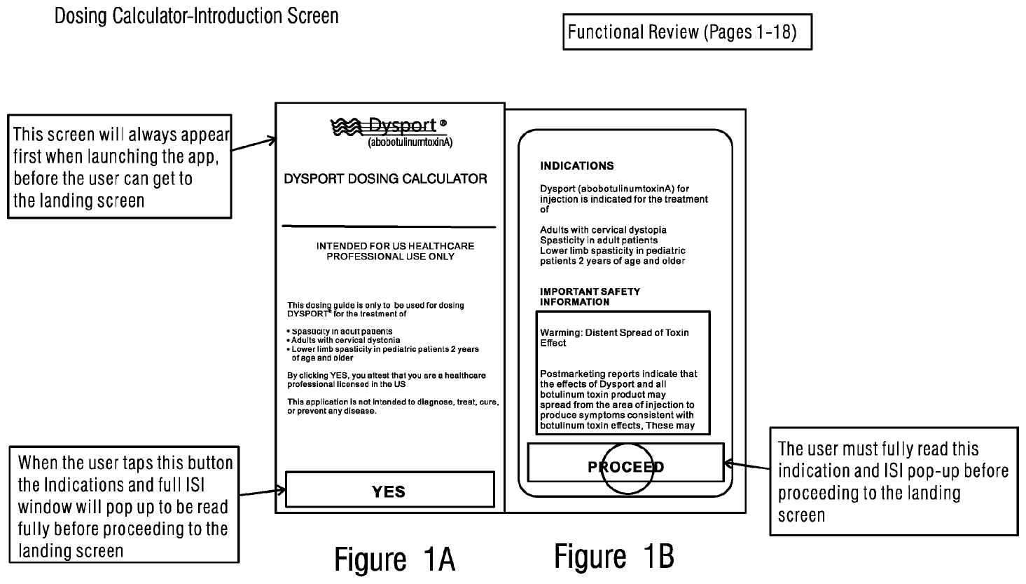

An embodiment of the present invention is illustrated by FIGS. 1-16 and by the following Example. It is understood that these Figures and the following Example are merely exemplary and are being used to illustrate the present invention. They do not serve to limit the scope of this invention.

### Example

A dosing calculator app for use in determining a dose amount of DYSPORT® for administration to a subject is provided.

The app provides a launch screen, illustrated in FIG. 1A. When the user agrees, by tapping on an affirmation button, to the use conditions for the app, the user may proceed to a rules/guidelines screen, such as is illustrated in FIG. 1B. It is required that the user scroll through all rules and guidelines, prior to tapping a button at the bottom of the screen.

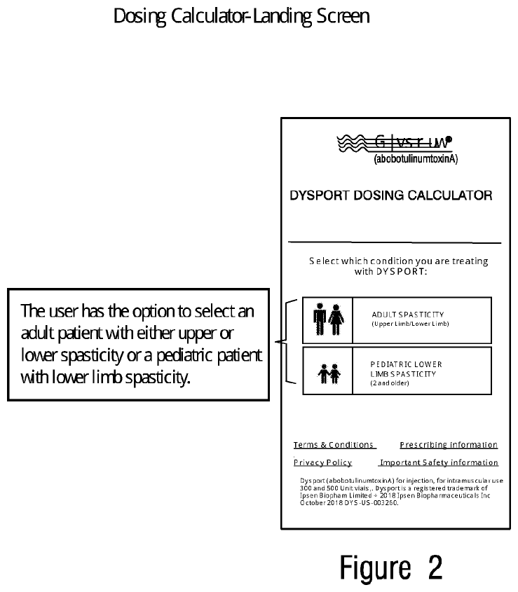

After the button is tapped, the user is taken to a landing screen (FIG. 2) whereon the user is enabled to select the disorder to be treated, in this case adult upper and/or lower limb spasticity or pediatric lower limb spasticity.

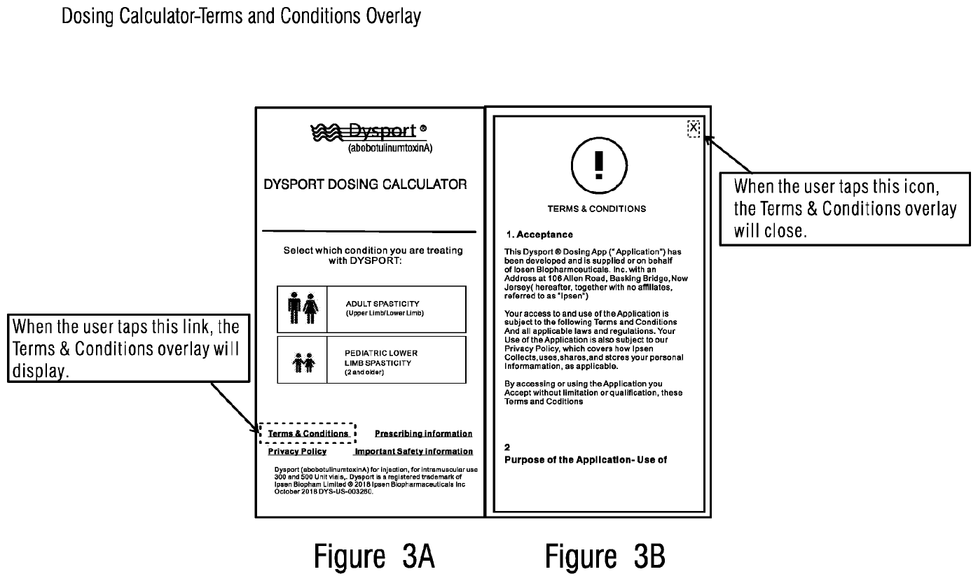

This screen also contains a link (FIG. 3A) which, when tapped, opens a display containing the terms and conditions (FIG. 3B). This display contains an icon at the upper right which, when tapped, closes the display and thus takes the user back to the landing screen.

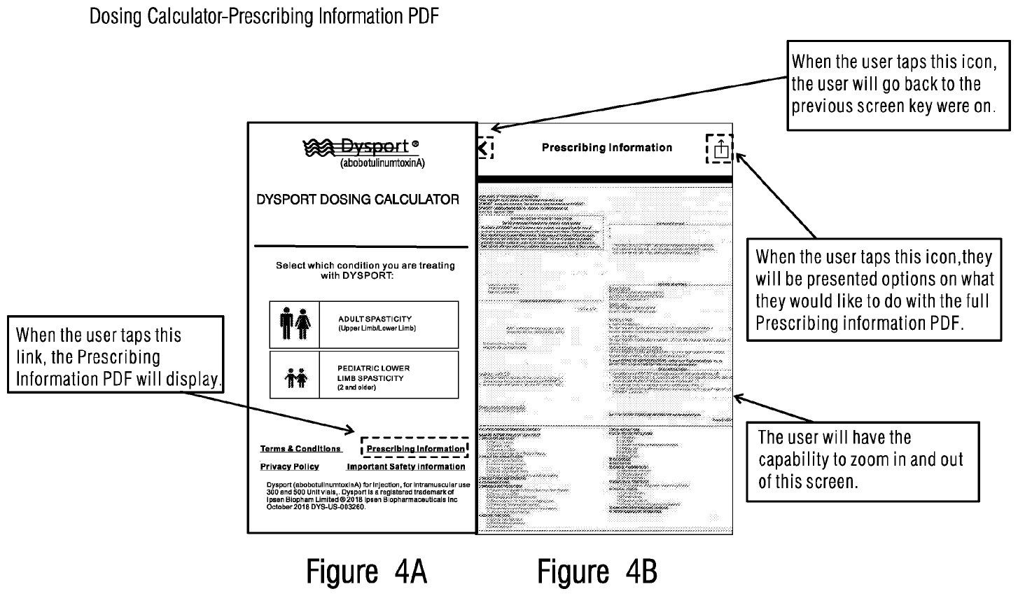

A link (FIG. 4A) is also provided which, when tapped, opens a display containing the prescribing information (FIG. 4B). This display is a PDF which the user can zoom in and out of. At the upper right corner of the display, an icon is present (id.). When tapped, the user will be presented with options on what they would like to do with the PDF. The options include printing the PDF or sending the PDF by e-mail. At the upper left corner of the display is a back button which, when tapped, sends the user back to the landing screen described previously.

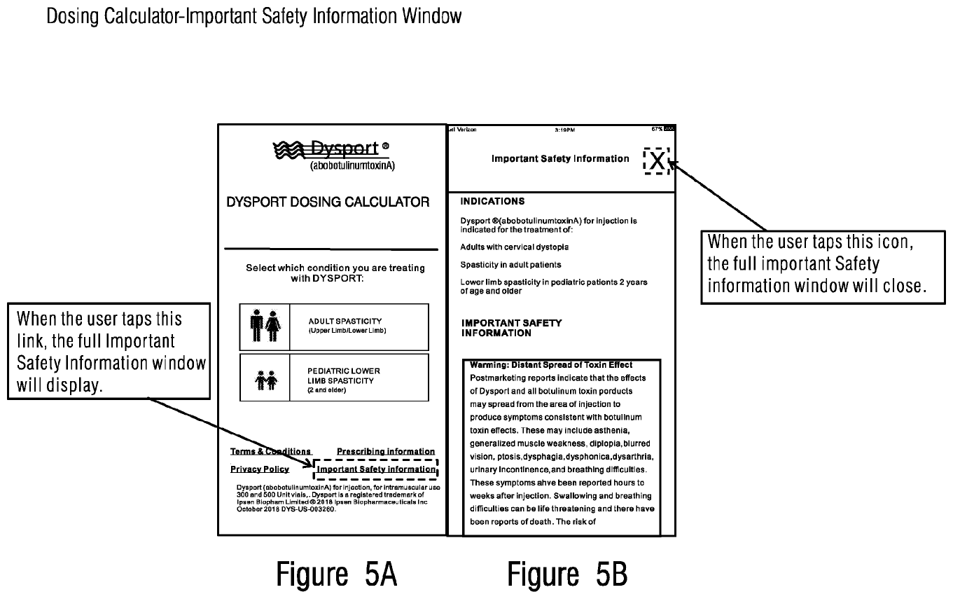

Similarly, a link (FIG. 5A) is provided which, when tapped, opens a display containing safety information (FIG. 5B). At the upper right corner of the display, an icon is present which, when tapped, closes the display and returns the user to the previous landing screen.

The privacy policy is similarly accessible by a link on the landing screen.

The user may select the disorder to be treated by tapping a button on the landing screen (FIG. 6A). Upon such selection, the user is taken to the “Step 1” screen (FIG. 6B). In the present example, the selected disorder is pediatric lower limb spasticity.

On this screen, the user is presented with buttons for entry of the subject's age and weight. When the user taps on the button for the subject's age (FIG. 7A), a widget appears (FIG. 7B). The user may use the widget to select the subject's age. Similarly, when the user taps on the button for the subject's weight (FIG. 8A), a widget appears (FIG. 8B) which the user may use to select the subject's weight.

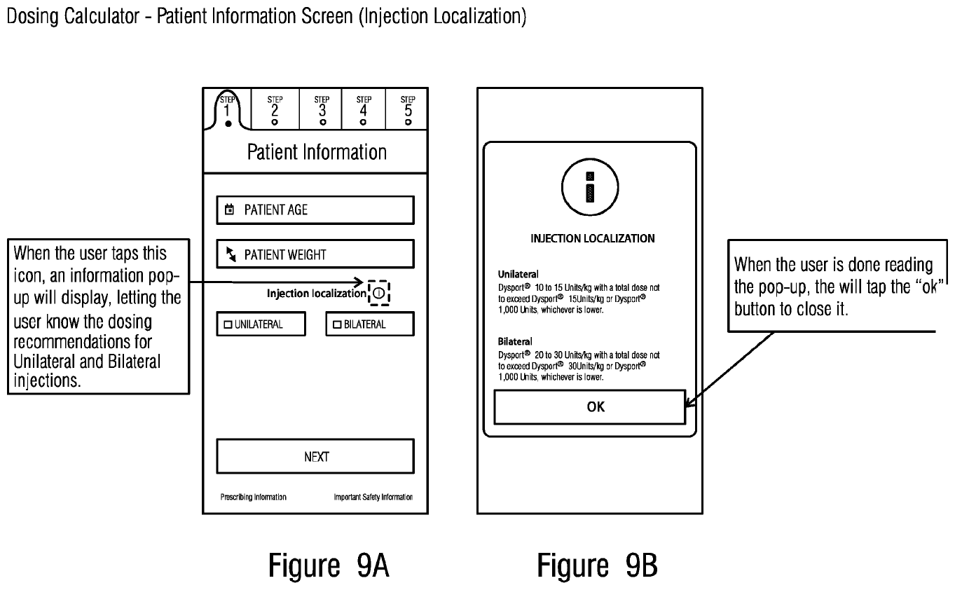

This screen also contains an icon (FIG. 9A) which, when tapped, opens a display containing information regarding the injection of DYSPORT® (FIG. 9B).

This screen further contains buttons which the patient may tap to indicate either unilateral administration or bilateral administration (FIG. 10A).

Based on the information previously input, the app recommends a maximum dose for the subject, which appears on the screen (id.)

At the bottom of the “Step 1” screen is a button which, when tapped, advances the user to the “Step 2” screen (FIG. 10B).

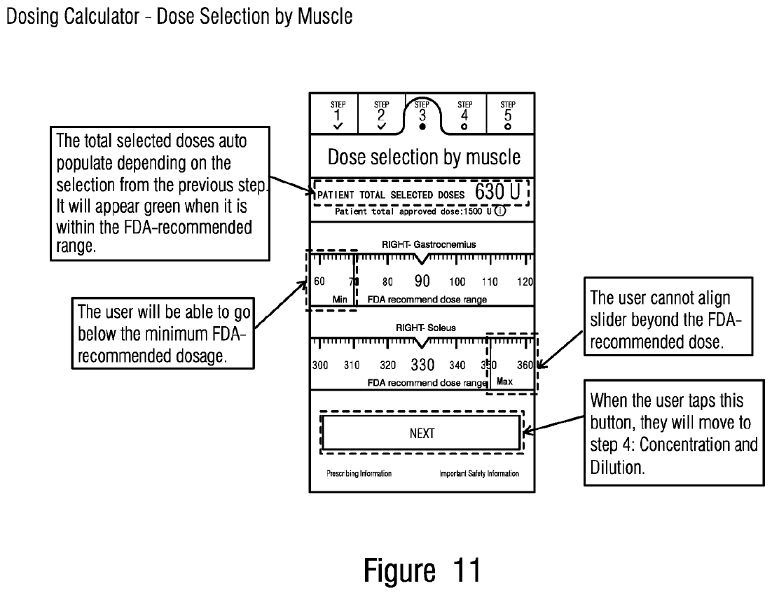

In the present example, the user selected bilateral administration on the “Step 1” screen. As such, the “Step 2” screen contains buttons for selecting which gastrocnemius (left or right) and which soleus (left or right) muscle to which DYSPORT® is to be administered. The user selects the appropriate muscle by tapping on the appropriate button. At the bottom of the screen is a button which, when pressed, advances the user to the “Step 3” screen (FIG. 11).

The total dose amount based on the previous entries is determined and is displayed on the “Step 3” screen (id.).

This screen also provides two sliding scales which the user may use to indicate the desired dosage at each location (id.). The user is precluded from adjusting the scales to indicate doses exceeding the FDA's recommended dose.

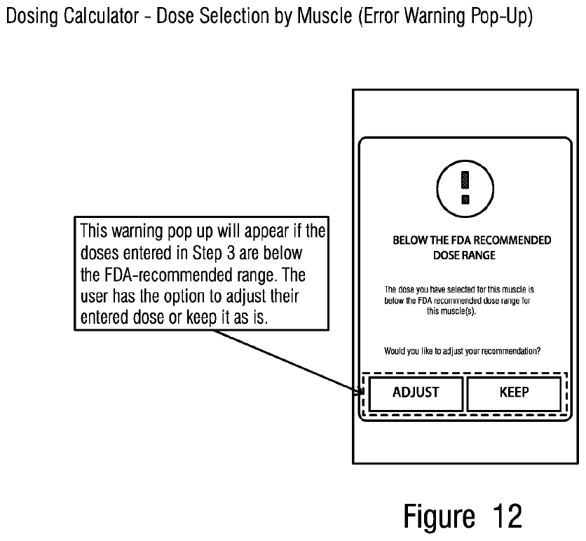

The user may, however, select a dose below the minimum FDA-recommended dosage. Although such a dose may not harm the patient, the selection of such a dose will trigger the appearance of a display (FIG. 12) providing a warning that the dose amount selected is below the FDA-recommended dose range. The display contains two buttons which the user may press to indicate, alternatively, whether he/she would like to go back to the “Step 3” screen and re-adjust the dose amounts selected or to keep the selected dose amounts (id.). Upon tapping either button, the user is returned to the “Step 3” screen.

At the bottom of the “Step 3” screen is a button which, when tapped, advances the user to the “Step 4” screen (FIG. 11).

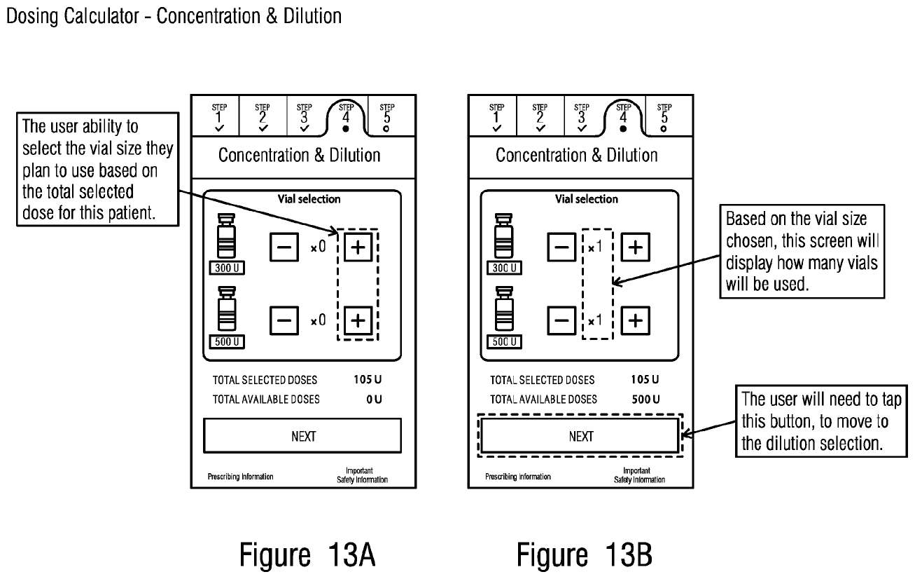

The “Step 4” screen contains buttons which allow the user to select the size of vial (300 U or 500 U) to be used when administering DYSPORT® (FIG. 13A). Based on the size chosen, the number of such vials to be used is determined and displayed on the screen (FIG. 13B).

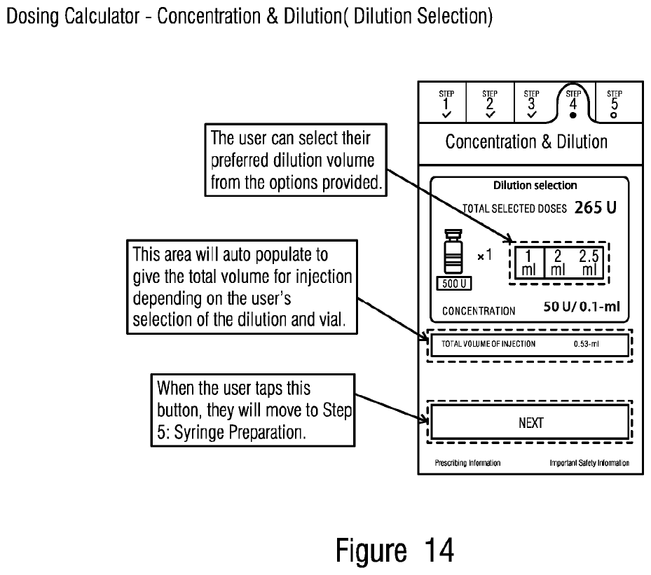

At the bottom of the screen is a button which, when pressed, changes the screen to display buttons for selecting a desired dilution volume (FIG. 14). The total volume for injection is then determined and displayed on the screen. The user may then press the button at the bottom of the screen again (id.). Upon tapping the button, the user is advanced to the “Step 5” screen (FIG. 15).

The “Step 5” screen contains buttons which the user may tap to indicate the type of syringe from which DYSPORT® will be administered (id.). Upon tapping such a button, a visual is provided on the screen to show what a syringe filled with the appropriate amount of the DYSPORT® preparation will look like (id.).

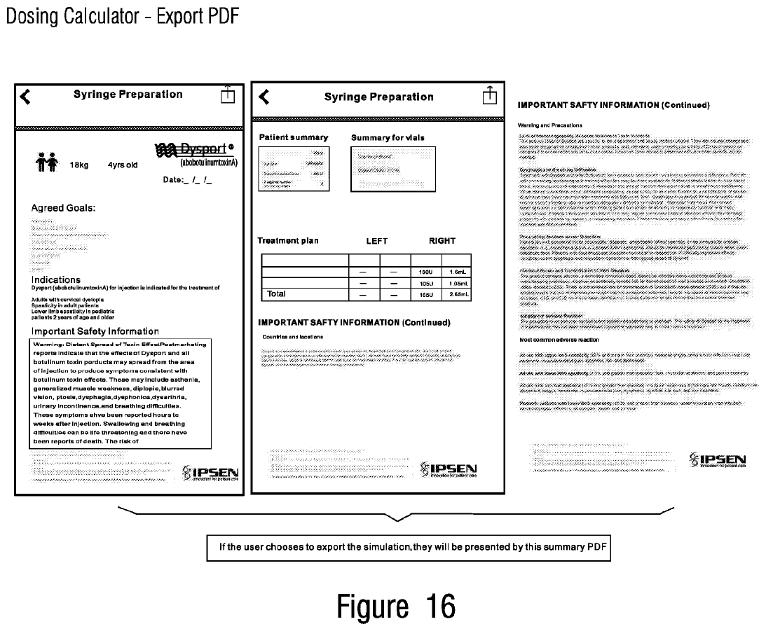

At the bottom of the screen is a button which, when tapped, will generate a PDF summarizing the information entered, options chosen, and the dosing determinations made (the “simulation”) (FIG. 16). This PDF may be printed or forwarded, for example by e-mail.

Also at the bottom of the screen is a button which, when tapped, will return the user to the “Step 1” screen to allow him/her to restart the dosing calculation by entering different parameters (i.e., start a new simulation) (FIG. 15)

What is claimed is:

1. A mobile computing application for use in the determination of a recommended dose of a medicinal for use in treating a disorder in a subject, wherein information is input by a user and the total dose for administration to the subject is determined.

2. The application of claim 1, wherein the information input by the user relates to the subject, the medicinal to be administered, and/or the location(s) for the administration.

3. The application of any one of claims 1 or 2, wherein administration is at more than one location and dose amounts for each location are determined.

4. The application of any one of claims 1-3, wherein administration is at more than one location, the user inputs a desired dose amount for a first location and the recommended dose amount for a second location is determined.

5. The application of any one of claims 1-4, wherein the information input by the user includes the container used for the administration of the medicinal and, based on such information, the number of such containers needed for the administration of the medicinal is determined.

6. The application of any one of claims 1-5, wherein the information input by the user includes the dilution for the medicinal and, based on such selection, the total volume of the medicinal formulation for administration is determined.

7. The application of any one of claims 1-6, wherein the medicinal is a neurotoxin.

8. The application of any one of claims 1-7, wherein the disorder is spasticity.

9. The application of any one of claims 1-8, wherein the disorder is upper or lower limb spasticity and the medicinal is botulinum neurotoxin A.

10. The application of any one of claims 1-9, wherein information regarding the recommended dose is accessible remotely by a third party.

11. A computer-implemented method for determining a dose of a medicinal for use in treating a disorder in a subject, wherein information is input by a user and the computer determines the total dose for administration.

12. The method of claim 11, wherein the information input by the user relates to the subject, the medicinal to be administered, and/or the location(s) for the administration.

13. The method of any one of claims 11 or 12, wherein administration is at more than one location and dose amounts for each location are determined.

14. The method of any one of claims 11-13, wherein administration is at more than one location and the user inputs a desired dose amount for a first location and the dose amount for a second location is determined.

15. The method of any one of claims 11-14, wherein a container used for the administration of the medicinal is selected and the number of such containers needed for such administration is determined.

16. The method of any one of claims 11-15, wherein a dilution for the medicinal is selected and, based on such selection, the total volume of the medicinal formulation for administration is determined.

17. The method of any one of claims 11-16, wherein the medicinal is a neurotoxin.

18. The method of any one of claims 11-17, wherein the disorder is spasticity.

19. The method of any one of claims 11-18, wherein the disorder is upper or lower limb spasticity and the medicinal is botulinum neurotoxin A.

20. A method for treating a disorder in a subject in need of such treatment, the method comprising using a computer-implemented method for determining a dose of the medicinal to be administered to the subject and administering such a dose to the subject.

21. The method of claim 20, wherein the disorder is a movement disorder.

22. The method of any one of claims 20 or 21, wherein the disorder is spasticity.

23. The method of any one of claims 20-22, wherein the medicinal is a neurotoxin.

24. The method of any one of claims 20-23, wherein the disorder is upper or lower limb spasticity and the medicinal is botulinum neurotoxin A.

25. The method of any one of claims 20-24, wherein the administration is intramuscular.

26. The method of any one of claims 20-25, wherein the determination of a dose of the medicinal is performed by a mobile computing application.
